(19) United States
(12) Patent Application Publication
(10) Pub. No.: US 2017/0319248 A1
(43) Pub. Date: Nov. 9, 2017

(54) VARIABLE-ANGLE LOCKING SCREWS, AND BONE PLATE SYSTEMS THAT INCLUDE VARIABLE-ANGLE LOCKING SCREWS

(71) Applicant: Cardinal Health 247, Inc., Dublin, OH (US)

(72) Inventors: MICHAEL JOSEPH MILELLA, JR., JOHNS CREEK, GA (US); SARAH ELIZABETH SCHAAKE, CENTENNIAL, CO (US)

(73) Assignee: Cardinal Health 247, Inc., Dublin, OH (US)

(21) Appl. No.: 15/148,456

(22) Filed: May 6, 2016

(51) Int. Cl. A61B 17/80; A61B 17/86; A61B 17/68

(52) U.S. Cl. CPC A61B 17/8014; A61B 17/8605; A61B 17/8057; A61B 2017/681

(57) ABSTRACT

A bone plate system may include a combination of one or more of a bone plate, non-locking screws, standard locking screws, and variable-angle locking screws. The bone plate may have threaded holes and non-threaded holes. Variable-angle locking screws may have a threaded shank and a threaded head. The threading on the head may be discontinuous, which allows the variable-angle locking screw to be inserted into a threaded hole on the plate at an angle relative to the axis of the threaded hole.

### BACKGROUND

Bone plates are used to provide structural support for fixation of various types of bone fractures. Bone plates are secured to the bone to “reduce” the fracture, i.e., bring bone fragments into alignment and close proximity to facilitate the body's natural bone growth and healing. Bone reduction is generally accomplished using non-locking screws. Non-locking screws have threaded shafts and are anchored through holes in the bone plate and into the various bone fragments, which upon tightening pull the bone fragments together under a compression load against the plate. However, due to dynamic loading caused by physiological movement, the non-locking screws may back out over time.

Locking screws may be anchored both to the bone plate and to the bone to reduce the incidence of loosening and provide a fixed angular relationship between the bone plate and the locking screws. A locking screw has a threaded head which mates with corresponding threads of a threaded hole in the bone plate, and a threaded shaft which anchors to the bone. Thus, because a locking screw is secured to both the bone and the bone plate, movement between the bone plate and the locking screws is reduced. As the relationship between the locking screws and the bone plate is fixed, locking screws provide a high resistance to shear or torsional forces. However, locking screws have a limited capability to compress bone fragments.

Thus, a combination of non-locking and locking screws may be employed with a bone plate to promote reduction of bone fractures while also preventing loosening and movement between the bone plate and the screws. Accordingly, bone plates may be configured with a combination of threaded and non-threaded holes. Standard locking screws may be inserted into a threaded hole at a fixed angle. However, it may be desirable to have a locking screw that is able to be inserted into the threaded hole at different angles.

### SUMMARY

Described herein is a variable-angle locking (VAL) screw insertable into a threaded hole, the VAL screw comprising a head having a first end, a second end, and an outer surface extending between the first end and the second end, and a shank extending from the first end of the head along a longitudinal axis of the VAL screw, wherein the outer surface of the head includes a plurality of thread segments, wherein the outer surface of the head includes engaging portions that engage the threaded hole and non-engaging portions that do not engage the threaded hole, and wherein the plurality of thread segments extend across the engaging portions and do not extend across the non-engaging portions.

A bone plate system is also described, the system comprising a plate having a threaded hole, wherein the threaded hole comprises a substantially continuous thread extending about an axis of the threaded hole, and a variable-angle locking (VAL) screw comprising a head having a first end, a second end, and an outer surface extending between the first end and the second end, and a shank extending from the first end of the head along a longitudinal axis of the VAL screw, wherein the outer surface of the head includes a plurality of thread segments, wherein the outer surface of the head includes engaging portions that engage the threaded hole and non-engaging portions that do not engage the threaded hole, wherein the plurality of thread segments extend across the engaging portions and do not extend across the non-engaging portions, wherein the VAL screw is insertable into the threaded hole of the plate.

Also described herein is a variable-angle locking (VAL) screw insertable into a threaded hole, the VAL screw comprising a head having a first end, a second end, and an outer surface extending between the first end and the second end, and a shank extending from the first end of the head along a longitudinal axis of the VAL screw, wherein the outer surface of the head comprises one or more discontinuous threads.

A bone plate system is also described, the system comprising a plate having a threaded hole, wherein the threaded hole comprises a substantially continuous thread extending about an axis of the threaded hole, and a variable-angle locking (VAL) screw comprising a head having a first end, a second end, and an outer surface extending between the first end and the second end, and a shank extending from the first end of the head along a longitudinal axis of the VAL screw, wherein the outer surface of the head comprises one or more discontinuous threads, and wherein the VAL screw is insertable into the threaded hole of the plate.

### BRIEF DESCRIPTION OF THE FIGURES

FIG. 1 shows a perspective view of a plating system, where a variable-angle locking screw is inserted, at an angle, into a threaded hole on a plate.

FIG. 2 shows a cross-sectional view of the plating system of FIG. 1, taken along line 2.

FIG. 3 shows a detailed cross-sectional view of the plating system of FIG. 1.

FIG. 4 shows a perspective view of the plating system of FIG. 1, except the variable-angle locking screw is coaxially inserted into the threaded hole on the plate.

FIG. 5 shows a cross-sectional view of the plating system of FIG. 4, taken along line 5.

FIG. 6 shows a detailed cross-sectional view of the plating system of FIG. 4.

FIG. 7 shows a perspective view of a bone plate used in the plating system of FIG. 1.

FIG. 8 shows a cross-sectional view of the plate of FIG. 7, taken along line 8.

FIG. 9A shows a detailed cross-sectional view of the plate of FIG. 7.

FIG. 9B shows a detailed cross-sectional view of the plate of FIG. 7.

FIG. 10 shows a perspective view of a variable-angle locking screw used in the plating system of FIG. 1.

FIG. 11 shows a side view of the variable-angle locking screw of FIG. 10.

FIG. 12 shows a front view of the variable-angle locking screw of FIG. 10.

FIGS. 13A-B show detailed front views of the variable-angle locking screw of FIG. 10.

FIG. 14 shows a top view of the variable-angle locking screw of FIG. 10.

FIG. 15 shows a perspective view of a standard locking screw used in the plating system shown in FIG. 16.

FIG. 16 shows a perspective view of a plating system.

FIG. 17 shows a side view of the plating system of FIG. 16.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details.

This disclosure uses various terms relating to threads on screws and on threaded holes. When an external surface of an item is threaded, this may be referred to as a male thread. On a male thread, the root is the point that is closest to the longitudinal axis of the thread, and the crest is the point that is farthest from the longitudinal axis of the thread. The major diameter is calculated by doubling the distance from the longitudinal axis to the crest. The minor diameter is calculated by doubling the distance from the longitudinal axis to the root. These distances are measured on a plane perpendicular to the longitudinal axis of the thread, which may also be referred to as a gauge plane.

When an internal surface of an object is threaded, this may be referred to as a female thread. On a female thread, the crest is the point that is closest to the longitudinal axis of the thread, and the root is the point that is farthest from the longitudinal axis of the thread. The major diameter is calculated by doubling the distance from the longitudinal axis to the root. The minor diameter is calculated by doubling the distance from the longitudinal axis to the crest. These distances are measured on a plane perpendicular to the longitudinal axis of the hole, which may also be referred to as a gauge plane.

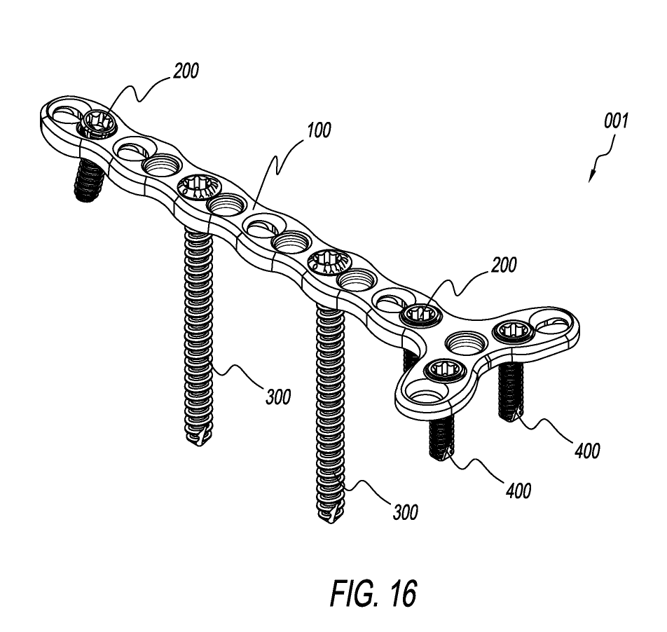

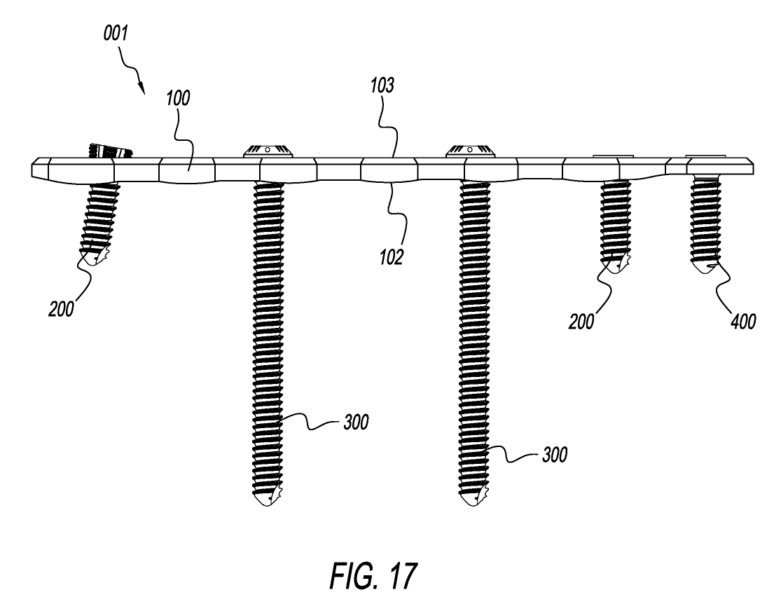

Internal fixation of bone fractures may be achieved using a plating system 001 shown in FIGS. 16-17. The plating system may include a plate 100, one or more non-locking screws 300, and one or more locking screws 200, 400. The locking screws included in the plating system 001 may be standard locking screws 400, variable-angle locking screws 200, or a combination thereof. The plate 100 may be used to bridge the fracture and temporarily provides fixation as the bone heals, and may be attached to the bone by one or more screws. Non-locking screws 300 may be used to provide tension or compression by pushing the plate 100 and bone fragments relative to one another. Non-locking screws 300 may also be used neutrally to buttress the fracture and hold fragments in place, but they may back out of the hole over time. Standard locking screws 400 and/or variable-angle locking screws 200 may be used to anchor the plate 100 to the bone or hold small bone fragments in place. The exemplary embodiment shown in FIGS. 16-17 includes a plate 100, two non-locking screws 300, two standard locking screws 400, and two variable-angle locking screws 200. However, any number of non-locking screws 300, standard locking screws 400, and variable-angle locking screws 200 may be used. Furthermore, the standard locking screws 400 are optional, as variable-angle locking screws 200 may be used in their place.

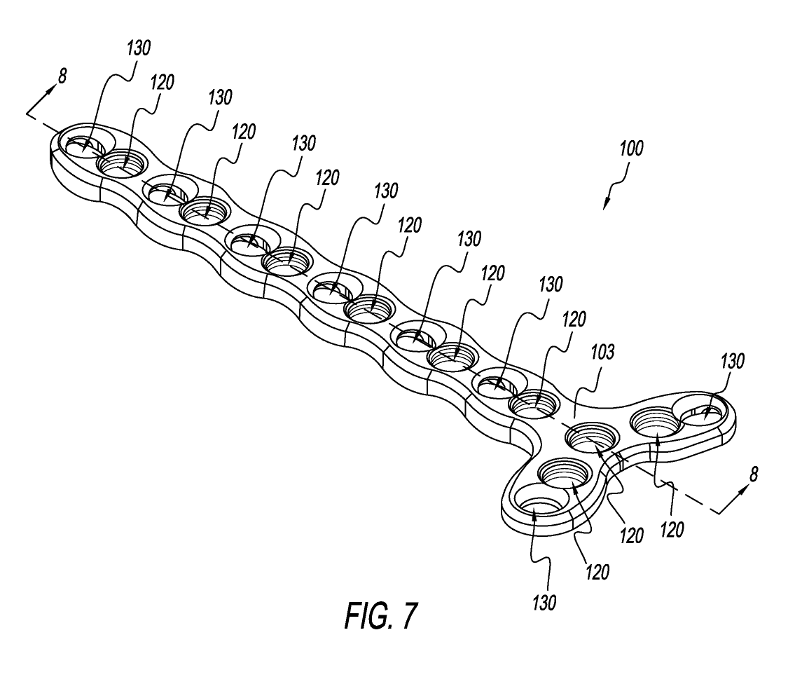

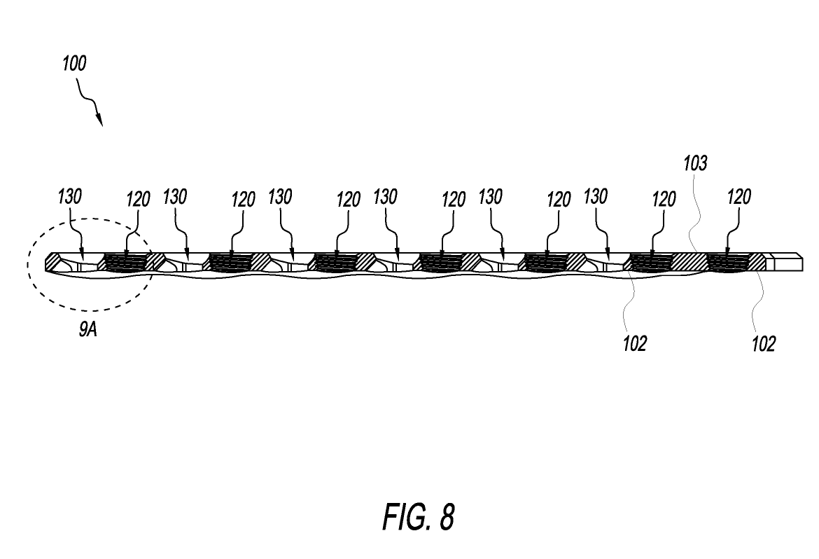

A bone plate, such as the plate 100 shown in FIGS. 7-9, may be used as part of an internal fixation system 001 for bone fractures. The bone plate 100 may be fabricated in any number of sizes and configurations in order to provide plates for use on different types of fractures. The plate 100 may be substantially planar, or it may be curved, angled, or otherwise formed to align with the patient's anatomy. The plate 100 may have a first surface 102 that faces the bone and a second surface 103 opposite the first surface 102.

The plate 100 may include one or more holes 120, 130. The holes 120, 130 may extend between the first surface 102 and the second surface 103. Some of the holes may be threaded holes 120, while other holes may be non-threaded holes 130. Typically, locking screws 200, 400 may be inserted into a threaded hole 120 and non-locking screws 300 may be inserted into a non-threaded hole 130.

One or more threaded holes 120 may be included on the plate 100. Each threaded hole 120 may have a first opening 122 on the first surface 102 of the plate 100 and a second opening 123 on the second surface 103 of the plate 100. An axis 124 of the threaded hole 120 may extend along a line passing through the center of the first opening 122 and the center of the second opening 123. Each threaded hole 120 may have one or more female threads 121. The one or more threads 121 may be substantially continuous.

The threaded hole 120 may be a tapered hole, or it may have another configuration. Preferably, the threaded hole 120 may be tapered such that the first opening 122 on the first surface 102 of the plate 100 is smaller than the second opening 123 on the second surface 103 of the plate 100. If the threaded hole 120 is tapered, each thread 121 may form a truncated conical helix following the taper of the threaded hole 120. Alternatively, the threaded hole 120 may be substantially straight (cylindrical), and each thread 121 may form a cylindrical helix.

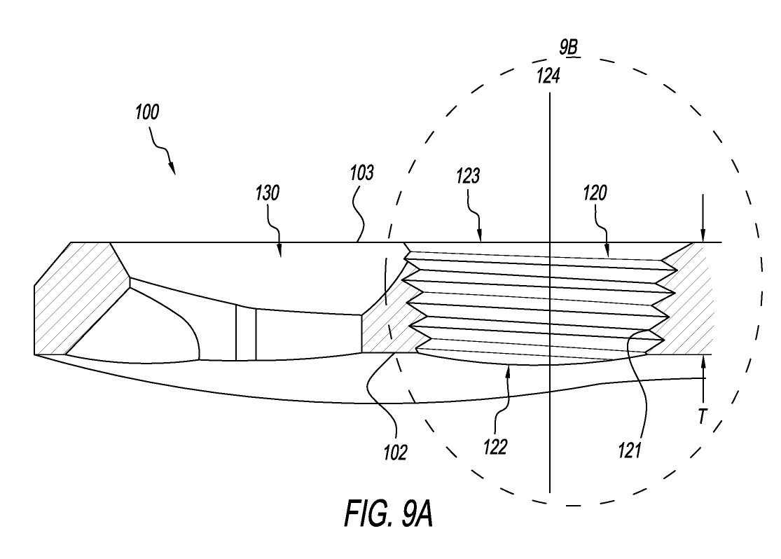

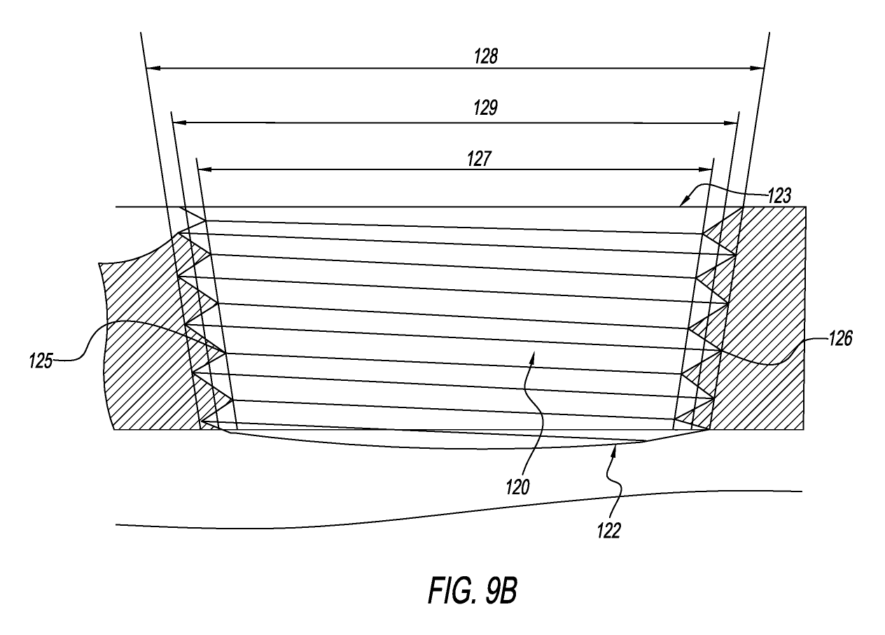

The one or more female threads 121 may form a series of alternating crests 126 and roots 125. The major diameter 128 of a thread 121 may be calculated by doubling the distance from the axis 124 of the threaded hole 120 to the root 125 of the thread 121. Likewise, the minor diameter 127 of a thread 121 may be calculated by doubling the distance from the axis 124 of the threaded hole 120 to the crest 126 of the thread 121. A theoretical major diameter may be calculated by extending the two edges of adjacent windings of the threads 121 until they intersect at a theoretical root, and doubling the distance from the axis 124 to the theoretical root to arrive at a theoretical major diameter. A theoretical minor diameter may also be calculated by extending the two edges of a thread 121 until they intersect at a theoretical crest, and doubling the distance from the axis 124 to this theoretical crest to arrive at a theoretical minor diameter. For the threaded hole 120 shown in FIG. 9B, the major diameter 128 is also the theoretical major diameter because two adjacent windings of the threads 121 intersect at a point to form the root 125. The minor diameter 127 is also to the theoretical minor diameter because the threads 121 have a triangular cross-section, so the two edges of a thread intersect at a point to form the crest 126. A pitch diameter may be calculated by taking the average of the theoretical major diameter and the theoretical minor diameter. In the threaded hole 120 shown in FIG. 9B, the pitch diameter 129 is the average of the minor diameter 127 and the major diameter 128.

The plate 100 may be manufactured using any materials that are commonly used in orthopedic fixation systems. For example, the plate 100 may be manufactured from a metal or polymeric material, or combinations thereof.

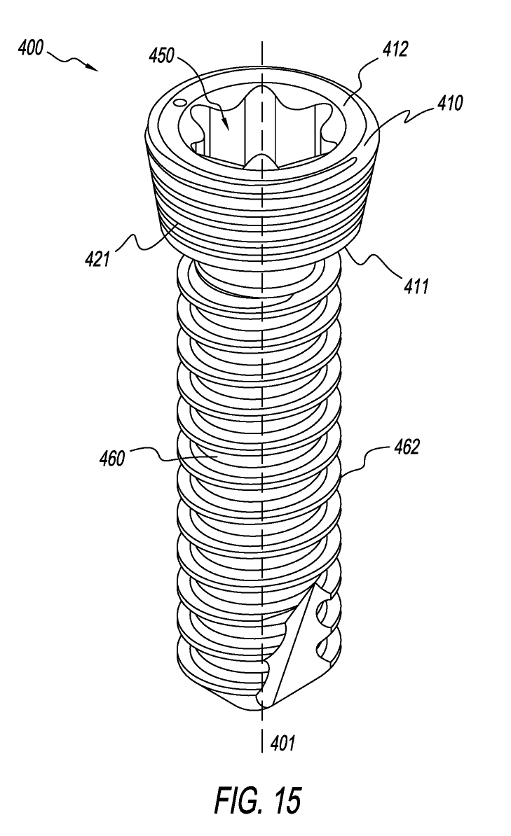

The system 001 may include one or more standard locking screws 400, shown in FIG. 15. A standard locking screw 400 may have a head 410 and a shank 460. The head 410 may have a first end 411, a second end 412, and an outer surface extending between the first end 411 and the second end 412. The shank 460 may extend from the first end 411 of the head 410 along a longitudinal axis 401 of the screw 400. A driving feature 450 may be included on the second end 412 of the head 410. The longitudinal axis 401 may intersect the center points of one or more of the first end 411 of the head 410, the second end 412 of the head 410, and the driving feature 450 of the head 410. Preferably, the longitudinal axis 401 may intersect the center points of all of the first end 411 of the head 410, the second end 412 of the head 410, and the driving feature 450 of the head 410.

The shank 460 of the standard locking screw 400 may include one or more male threads 462 extending along at least part of the outer surface of the shank 460. The threads 462 on the shank 460 allow the screw 400 to interlock with the bone. The threads 462 may extend substantially helically about the longitudinal axis 401 of the screw 400.

The outer surface of the head 410 may also include one or more male threads 421 which allow the screw 400 to interlock with a threaded hole 120 on the plate 100. The threads 421 on the outer surface of the head 410 of the standard locking screw 400 may be substantially continuous, such that the threads 421 fully engage the threads 121 on the threaded hole 120 of the plate 100.

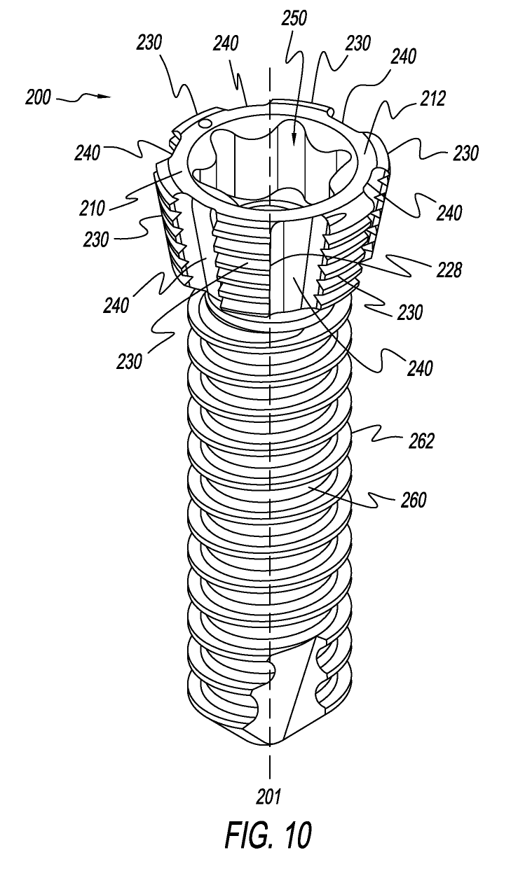

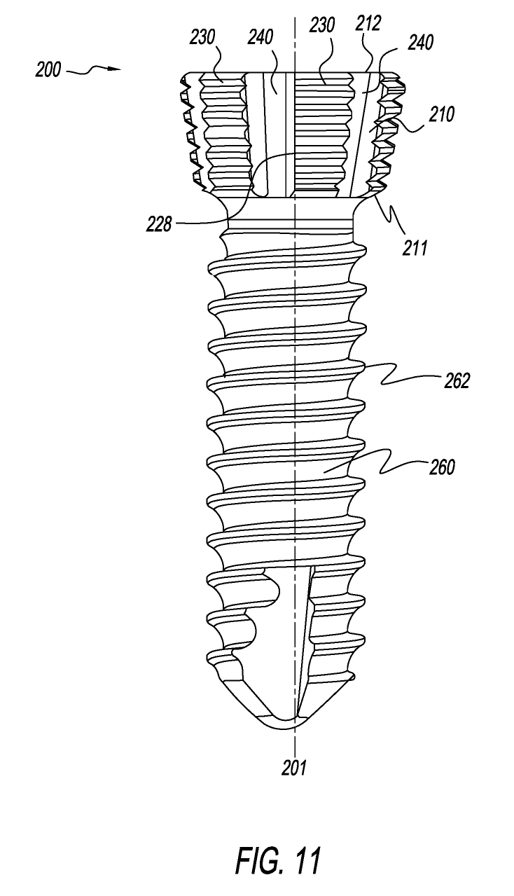

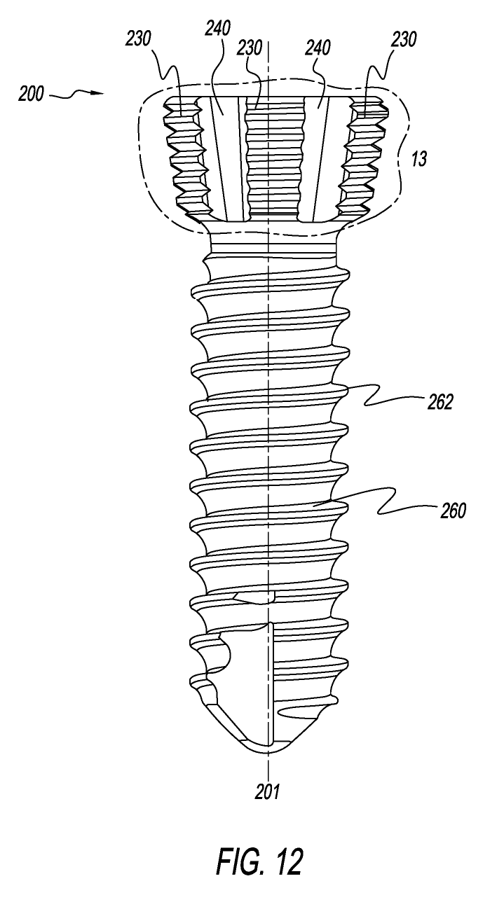

The system 001 may include one or more variable-angle locking screws 200, shown in FIGS. 10-14. A variable-angle locking screw 200 may have a head 210 and a shank 260. The head 210 may have a first end 211, a second end 212, and an outer surface extending between the first end 211 and the second end 212. The shank 260 may extend from the first end 211 of the head 210 along a longitudinal axis 201 of the screw 200. A driving feature 250 may be included on the second end 212 of the head 210. The longitudinal axis 201 may intersect the center points of one or more of the first end 211 of the head 210, the second end 212 of the head 210, and the driving feature 250 of the head 210. Preferably, the longitudinal axis 201 may intersect the center points of all of the first end 211 of the head 210, the second end 212 of the head 210, and the driving feature 250 of the head 210.

The shank 260 of the variable-angle locking screw 200 may include one or more male threads 262 extending along at least part of the outer surface of the shank 260. The threads 262 on the shank 260 allow the screw 200 to interlock with the bone. The threads 262 may extend substantially helically around the longitudinal axis 201 of the screw 200.

The outer surface of the head 210 may also include one or more male threads 221 which allow the variable-angle locking screw 200 to interlock with a threaded hole 120 on the plate 100. Unlike standard locking screws 400 which have substantially continuous threads 421, the threads 221 on the head 210 of the variable-angle locking screw 200 may be discontinuous, such that each thread 221 is separated into a plurality of thread segments 220.

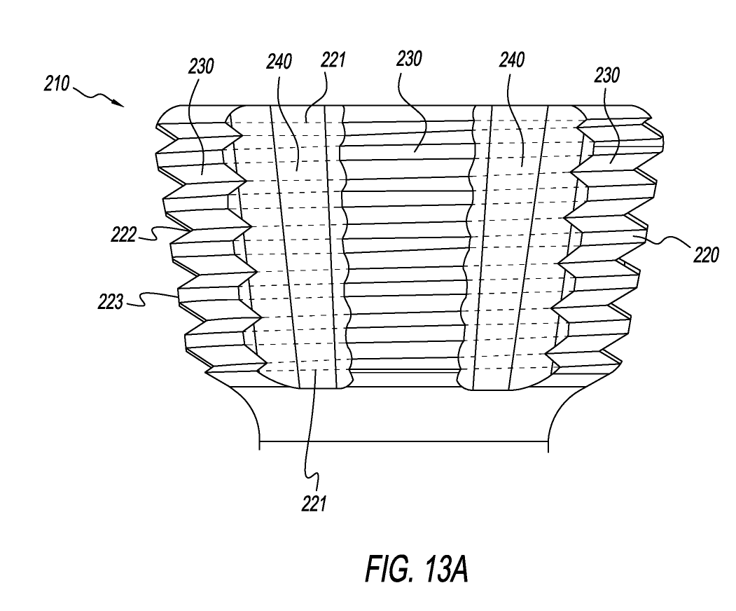

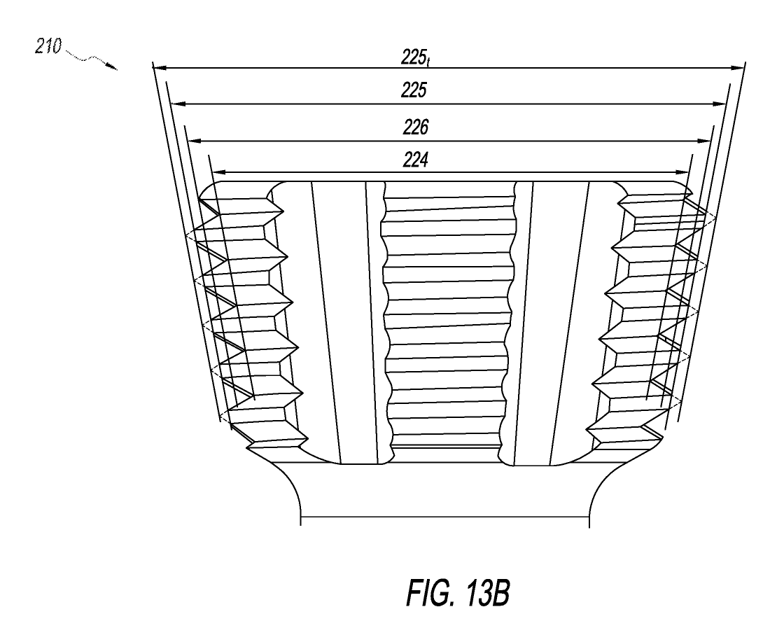

The head 210 may have engaging portions 230 that interlock with the threaded hole 120 of the plate 100, and non-engaging portions 240 which do not interlock with the threaded hole 120 of the plate 100. Each engaging portion 230 comprises at least one thread segment 220, and may further comprise a series of thread segments 220. When the variable-angle locking screw 200 is inserted into a threaded hole 120 of a plate 100, the thread segments 220 at the engaging portions 230 of the head 210 interlock with the threads 121 on the threaded hole 120 of the plate 100. The thread segments 220 of the engaging portions 230 do not extend across the non-engaging portions 240, and therefore the non-engaging portions 240 do not interlock with the threads 121 on the threaded hole 120 of the plate 100. The non-engaging portions 240 may extend from the first end of the head to the second end of the head. The engaging portions 230 and non-engaging portions 240 may alternate around the outer surface of the head 210, forming alternating columns around the outer surface of the head 210. Each column may be formed by some of the thread segments 220. The thread segments 220 on the engaging portions 230 may be aligned such that, if the thread segments 220 were extended across the non-engaging portions 240, they would form substantially continuous threads 221 extending helically around the head 210, as shown by the dashed lines in FIG. 13A. In other words, a curve extends along a trajectory formed by a crest 223 of a first thread segment 220 on a first engaging portion 230, and the curve follows the trajectory across a non-engaging portion 240 and also extends along a trajectory formed by a crest 223 of a second thread segment 220 on an adjacent engaging portion 230.

Each engaging portion 230 of the head 210 of the variable-angle locking screw 200 may comprise a series of alternating crests 223 and roots 222 that form the thread segments 220. The number of thread segments 220 present in a particular engaging portion 230 may be counted by counting the number of crests 223 present in that engaging portion 230. Each engaging portion may have at least one crest 223. The engaging portions 230 may each have a substantially similar number of crests 223.

The minor diameter 224 of a thread 221 may be calculated by doubling the distance from the longitudinal axis 201 to the root 222 of the thread 221. Likewise, the major diameter 225 of a thread 221 may be calculated by doubling the distance from the longitudinal axis 201 to the crest 223 of the thread 221. A theoretical major diameter (225ₜ) may be calculated by extending the two edges of a thread segment 220 until they intersect at a theoretical crest (as shown in FIG. 13B), and doubling the distance from the longitudinal axis 201 to this theoretical crest to arrive at a theoretical major diameter. A theoretical minor diameter may also be calculated by extending the edges of two adjacent thread segments 220 until they intersect at a theoretical root, and doubling the distance from the longitudinal axis 201 to this theoretical root to arrive at a theoretical minor diameter. For the screw 200 shown in FIG. 13B, the minor diameter 224 is also the theoretical minor diameter because two adjacent thread segments 220 intersect at a point to form the root 222. However, the major diameter 225 is not equal to the theoretical major diameter (225ₜ) because the thread 221 has a trapezoidal cross-section. A pitch diameter may be calculated as the average of the theoretical major diameter and the theoretical minor diameter. In the screw shown in FIG. 13B, the pitch diameter 226 is the average of the minor diameter 224 and the theoretical major diameter (225ₜ).

The non-engaging portions 240 may be formed in a variety of shapes. Typically, the non-engaging portions 240 are substantially unthreaded. In some embodiments, the surface of the non-engaging portions 240 may follow the shape of the head 210 of the variable-angle locking screw 200 to minimize the amount of material that is removed from the screw 200 to create the non-engaging portions 240. For example, if the head 210 is tapered, then the surface of the non-engaging portion 240 may also be tapered. Alternatively, the non-engaging portions 240 may be substantially planar surfaces that extend between the adjacent engaging portions 230.

The border 228 between an engaging portion 230 and an adjacent non-engaging portion 240 may be formed substantially along an intersection between the outer surface of the head 210 and a plane in which the longitudinal axis 201 lies. As such, each border 228 between an engaging portion 230 and an adjacent non-engaging portion 240 may be coplanar with the longitudinal axis 201.

The following equation may be used to determine the angulation of the variable-angle locking screw 200 in the threaded hole 120 of the plate 100.

$$x = ( y - H ) \cdot ( \frac{{P}_{hole}}{T \cdot R \cdot ( \frac{{P}_{screw} \cdot {N}_{seg} \cdot {θ}_{seg}}{360} )} ) \\ x = screw angle \\ H = nominal screw head protrusion height \\ y = screw head protrusion height \\ T = plate thickness \\ R = engagement ratio \\ {P}_{hole} = hole pitch diameter \\ {P}_{screw} = screw pitch diameter \\ {N}_{seg} = number of engaging portions \\ {θ}_{seg} = segment angle \qquad Equation 1$$

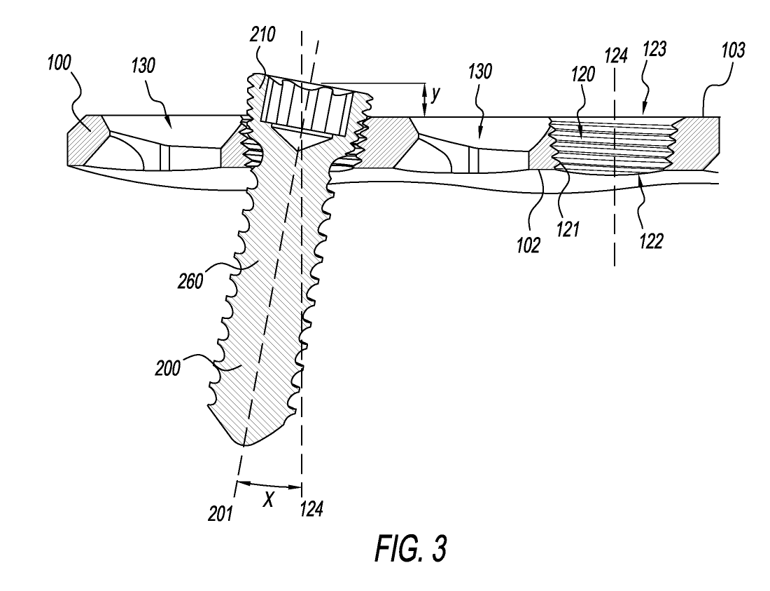

The screw angle (x), shown in FIG. 3, is the angle between the longitudinal axis 201 of the variable-angle locking screw 200 and the axis 124 of the threaded hole 120 of the plate 100. The screw angle (x) is greater than or equal to 0°. As the axis 201 of the screw 200 moves away from being coaxial with the axis 124 of the threaded hole 120, the screw angle (x) increases.

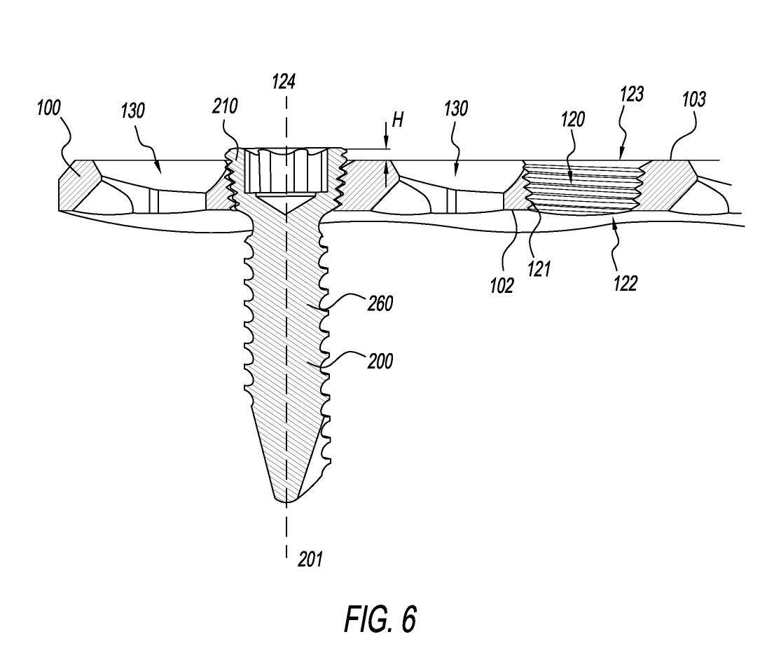

The nominal screw head protrusion height (H), shown in FIG. 6, is the distance between the center of the second end 212 of the head 210 of the variable-angle locking screw 200 and a plane containing the second opening 123 of the threaded hole 120 of the plate 100. The nominal screw head protrusion height (H) is measured when the screw 200 is inserted into the threaded hole 120 coaxially, such that the screw angle (x) is zero.

The screw head protrusion height (y), shown in FIG. 3, is the distance between the center of the second end 212 of the head 210 of the variable-angle locking screw 200 and a plane containing the second opening 123 of the threaded hole 120 of the plate 100. If the screw 200 is coaxially inserted into the threaded hole 120, then the head protrusion height (y) equals the nominal screw head protrusion height (H). The nominal screw head protrusion height (H) will be greater or equal to zero. In general, the screw head protrusion height (y) may increase as the screw angle (x) increases.

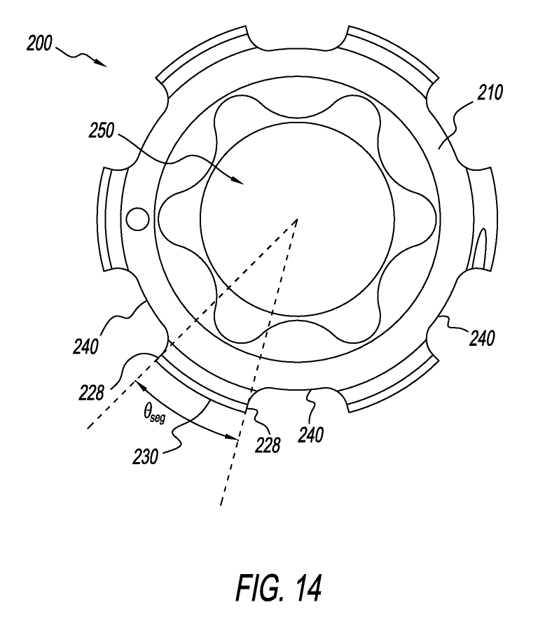

The segment angle (θ_{seg}), shown in FIG. 14, is the angle between the borders 228 on either side of an engaging portion 230 of the variable-angle locking screw 200, if the borders 228 were each extended to intersect the longitudinal axis 201 of the screw 200, as shown in FIG. 14.

The number of engaging portions (N_{seg}) is the total number of engaging portions 230 on the head 210 of the variable-angle locking screw 200.

The engagement ratio (R) is the number of engaging portions 230 having at least one thread segment 220 that engages the threaded hole 120 on the plate 100 divided by the total number of engaging portions (N_{seg}). The engagement ratio (R) may range from 0 (if none of the thread segments 220 are engaged with the threaded hole 120) to 1 (if each engaging portion 230 has at least one thread segment 220 on engaged with the threaded hole 120). Preferably, the engagement ratio (R) is greater than zero.

The hole pitch diameter (Pₕₒₗₑ) is the pitch diameter of the threaded hole 120 of the plate 100. If the threaded hole 120 of the plate 100 is not cylindrical, the hole pitch diameter (Pₕₒₗₑ) is measured on a gauge plane at the widest part of the threaded hole 120. For example, if the threaded hole 120 of the plate 100 forms a truncated cone, wherein the second opening 123 is larger than the first opening 122, as shown in FIG. 9A, then the hole pitch diameter (Pₕₒₗₑ) would be determined based on the theoretical minimum thread diameter and theoretical maximum thread diameter on a gauge plane at the second surface 103 of the plate 100. Increasing the hole pitch diameter (Pₕₒₗₑ) may allow for a greater screw angle (x).

The plate thickness (T) is the distance between the first surface 102 of the plate 100 and the gauge plane at the widest part of the threaded hole 120. In FIG. 9A, the plate thickness (T) is the distance between the first surface 102 of the plate 100 and the second surface 103 of the plate 100 adjacent the threaded hole 120 because the gauge plane is at the second surface 103 of the plate 100. Increasing the plate thickness (T) may limit the screw angle (x). If a thicker plate is desired, a counter bore or relief area may be added to the first opening 122 of the threaded hole 120 of the plate 100 to offset an increase in thickness.

The screw pitch diameter (P_{screw}) is the pitch diameter of the head 210 of the variable-angle locking screw 200. The screw pitch diameter (P_{screw}) may be measured on a gauge plane at the widest part of the head 210 of the screw 200. For example, if the head 210 of the screw 200 forms a truncated cone, wherein the second end 212 is larger than the first end 211, then the screw pitch diameter (P_{screw}) would be determined based on the theoretical minimum thread diameter and theoretical maximum thread diameter on a gauge plane at the second end 212 of the head 210 of the screw 200. Increasing the screw pitch diameter (P_{screw}) may limit the screw angle (x).

In Equation 1, the screw angle (x) and segment angle (θ_{seg}) are measured in degrees; however, the equation could be modified to accommodate other units of measurement, such as radians. The plate thickness (T), nominal screw head protrusion height (H), screw head protrusion height (y), hole pitch diameter (Pₕₒₗₑ), and screw pitch diameter (P_{screw}) may be measured in millimeters, inches, or any unit of distance, so long as the same unit is used for all of these variables (or the equation is modified to accommodate different units of measurements). The number of engaging portions (N_{seg}) and engagement ratio (R) do not have units.

Equation 1 assumes that, if the threaded hole 120 of the plate 100 and the head 210 of the screw 200 have a tapered conical shape, a similar taper angle is used on the threaded hole 120 of the plate 100 and the head 210 of the screw 200.

The variable-angle locking screw 200 may have a plurality of engaging portions 230 and a plurality of non-engaging portions 240. The number of engaging portions 230 (also referred to as (N_{seg}) in Equation 1) and the number of non-engaging portions 240 may vary based on the dimensions of the screw 200. In some embodiments, the screw 200 may have at least two engaging portions 230 and at least two non-engaging portions 240. In other embodiments, screw may have between two and ten engaging portions 230 and between two and ten non-engaging portions 240. In preferred embodiments, the screw 200 may have at least three engaging portions 230 and at least three non-engaging portions 240. In other preferred embodiments, screw may have between three and ten engaging portions 230 and between three and ten non-engaging portions 240.

Each engaging portion 230 and non-engaging portion 240 may cover a percentage of the outer surface of the head 210. The percentage of the outer surface of the head 210 covered by each engaging portion may be calculated as

$$( \frac{{θ}_{seg} \cdot 100}{360} ) .$$

In some embodiments, each engaging portion 230 may cover approximately the same percentage of the outer surface of the head 210. Each non-engaging portion 240 may cover approximately the same percentage of the outer surface of the head 210. However, the percentage of the outer surface of the head 210 covered by each engaging portion 230 is not necessarily the same as the percentage of the outer surface of the head 210 covered by each non-engaging portion 240.

The total percentage of the outer surface of the head 210 covered by all of the engaging portions 230 may be calculated by adding together the percentage of the outer surface of the head 210 that each engaging portion 230 covers. If each engaging portion 230 covers the same percentage of the outer surface of the head 210, then the total percentage of the outer surface of the head 210 covered by all of the engaging portions 230 combined may be calculated as

$$( \frac{{N}_{seg} \cdot {θ}_{seg}}{360} \cdot 100 ) .$$

The total percentage of the outer surface of the head 210 covered by all of the engaging portions 230 may vary. In some embodiments, the total percentage of the outer surface of the head 210 covered by all of the engaging portions 230 may be at least about 25%. In other embodiments, the total percentage of the outer surface of the head 210 covered by all of the engaging portions 230 may range from about 25% to about 99%. In other embodiments, the total percentage of the outer surface of the head 210 covered by all of the engaging portions 230 is at least half of the outer surface of the head 210 and less than the entire outer surface of the head 210. In other embodiments, the total percentage of the outer surface of the head 210 covered by all of the engaging portions 230 may be at least about 50%. In other embodiments, the total percentage of the outer surface of the head 210 covered by all of the engaging portions 230 may range from about 50% to about 99%. However, increasing the total percentage of the outer surface of the head 210 that is covered by the engaging portions 230 may limit the screw angle (x) that can be achieved.

The sum of the percentage of the outer surface covered by the engaging portions 230 and the percentage of the outer surface covered by the non-engaging portions 240 may equal 100%. Therefore, in some embodiments, the total percentage of the outer surface of the head 210 covered by all of the non-engaging portions 240 may be less than about 75%. In other embodiments, the total percentage of the outer surface of the head 210 covered by all of the non-engaging portions 240 may range from about 1% to about 75%. In other embodiments, the total percentage of the outer surface of the head 210 covered by all of the non-engaging portions 240 may be less than about 50%. In other embodiments, the total percentage of the outer surface of the head 210 covered by all of the non-engaging portions 240 may range from about 1% to about 50%.

In an exemplary, non-limiting example, the variable-angle locking screw 200 may have six engaging portions 230 and six non-engaging portions 240. Each engaging portion 230 may cover about 8.3% of the outer surface of the head 210. Therefore, when added together, the engaging portions 230 may cover a total of about 50% of the outer surface of the head 210. Each non-engaging portion 240 may cover about 8.3% of the outer surface of the head 210. Therefore, when added together, the non-engaging portions 240 may cover a total of about 50% of the outer surface of the head 210.

If the thread segments 220 were extended across the non-engaging portions 240 of the head 210, they may form one or more substantially continuous threads 221, each thread 221 being shaped as a helix around the longitudinal axis 201 of the variable-angle locking screw 200. Preferably, the head 210 may be tapered such that the head 210 is narrower near the first end 211 and wider near the second end 212. Accordingly, the threads 221 may also be tapered, such that each thread 221 forms a truncated conical helix.

The thread segments 220 on the head 210 of the variable-angle locking screw 200, may be arranged such that, if extended across the non-engaging portions 240 of the head 210, they would form any number of substantially continuous male threads 221. For example, the thread segments 220 may be arranged to create a single-start thread forming a single helix, if they were extended to form a substantially continuous thread 221. However, the thread segments 220, if extended, could also form multiple threads. The number of threads 221 formed on the head 210 of the screw (if the thread segments 220 were extended across the non-engaging portions 240) may be the same as the number of threads 121 on the threaded hole 120 in the plate 100. In some embodiments, both the threaded hole 120 in the plate 100 and the head 210 of the screw 200 may have double-start threads in which the threads 121, 221 form a double helix. In some embodiments, the threads 121, 221 may form in a truncated conical double helix.

A number of manufacturing techniques may be used to make a variable-angle locking screw 200. One manufacturing method may include obtaining a standard locking screw (with a substantially continuously-threaded head), and removing material from the outer surface of the head to form the non-engaging portions 240. Alternatively, the material at the non-engaging portions 240 may be removed from the outer surface of the head 210 and then the threads in the engaging portions 230 may be formed. The screw 200 may also be made using injection molding, 3D printing, or various other known techniques.

The system 001 may include various sizes of screws 200, 300, 400, with varying lengths and diameters. Any size of screw may be used. The screws may be sized appropriately for use in orthopedic fixation systems.

The screws 200, 300, 400 may be manufactured using any materials that are commonly used in orthopedic fixation systems. The screws 200, 300, 400 may be manufactured from a metal or polymeric material, or combinations thereof. The plate 100 and the locking screws 200, 400 may be manufactured from the same (or similar) materials having the same (or similar) properties, or they may be manufactured from different materials having different properties.

As discussed above, the locking screws 200, 400 may include a driving feature 250, 450. The non-locking screw 300 may also include a driving feature. The driving feature may be designed to receive a driving tool (for example, a screw driver, socket, or any other drive type known in the art) which can be used to drive the shank of the screw into the bone. In the case of locking screws 200, 400, the driving tool may also be used to thread the head 210, 410 of the screw into the threaded hole 120 in the plate 100. The driving feature may be a slot, cruciate, square, hex, hexalobe, or any other drive type known in the art. All of the screws 200, 300, 400 in the system 001 may use the same type of driving feature, or different driving features may be used on different screws.

A variety of cross-sectional shapes may be used to form the threads described in this application, including triangular, trapezoidal, rectangular, or a variety of other cross-sectional shapes. For example, any of the threads described in this application may have a triangular cross-sectional shape similar to the threads 121 on the threaded hole 120 of the plate 100 shown in FIG. 9B. Any of the threads described in this application may also have a trapezoidal cross-sectional shape similar to the threads 221 on the head 210 of the screw 200 in FIGS. 13A-13B.

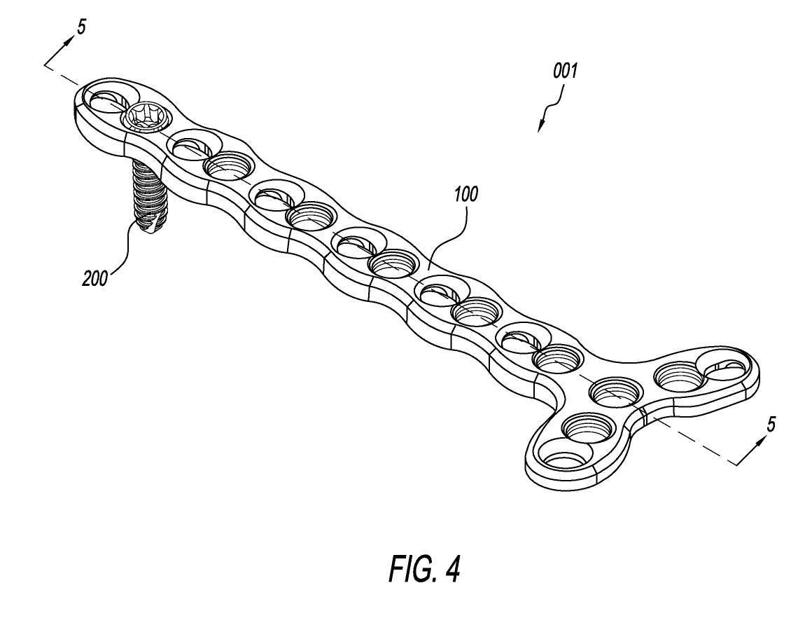

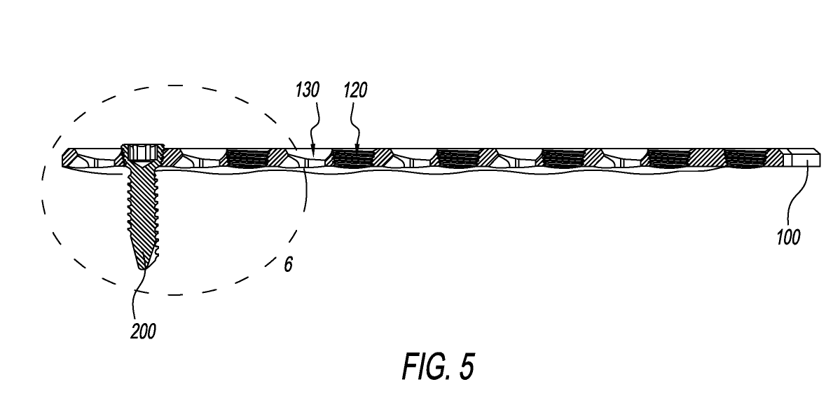

In some situations, a locking screw may be inserted coaxially into a threaded hole 120 of a plate 100 as shown in FIGS. 4-6. To achieve coaxial insertion, the surgeon may use either a variable-angle locking screw 200 or a standard locking screw 400. The longitudinal axis 201, 401 of the screw 200, 400 may be substantially parallel to the axis 124 of the threaded hole 120 on the plate 100. Using a standard locking screw 400 for coaxial insertion into the threaded hole 120 on the plate 100 may provide increased engagement between the screw and the plate because both the head 410 of the standard locking screw 400 and the threaded hole 120 on the plate 100 may have substantially continuous threads 421, 121. However, standard locking screws 400 are optional in system 001 because the threaded holes 120 on the plate 100 are able to interchangeably receive the variable-angle locking screw 200 and the standard locking screw 400. Variable-angle locking screws 200 may also be inserted coaxially into threaded holes 120 on the plate 100.

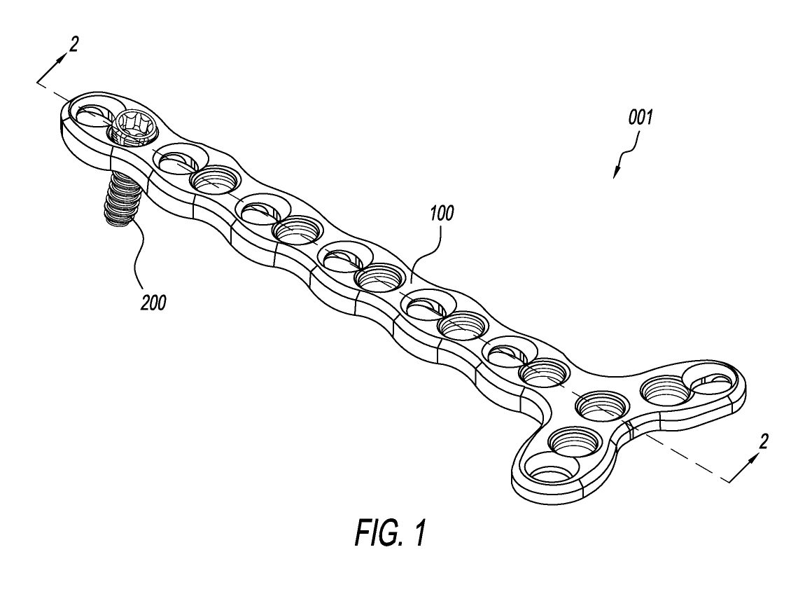

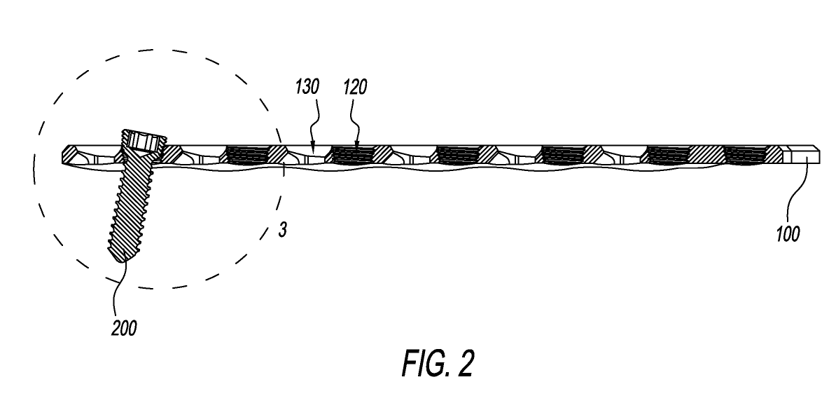

In other situations, instead of inserting the locking screw coaxially, a surgeon may wish to insert a locking screw into a threaded hole 120 of a plate 100 at an angle as shown in FIGS. 1-3. In this case, a variable-angle locking screw 200 may be inserted into the threaded hole 120 on the plate 100 at an angle to the axis 124 of the threaded hole 120. The screw 200 may be inserted into the threaded hole 120 on the plate 100 such that the screw angle (the angle between the longitudinal axis 201 of the screw 200 and the axis 124 of the threaded hole 120) may vary from about 0° (substantially coaxial) to about 20° or more in any direction, thereby forming a theoretical, substantially cone-shaped region in which the longitudinal axis 201 of the screw 200 may lie. FIGS. 1-3 show an exemplary configuration of the system 001 wherein a variable-angle locking screw 200 is inserted into a threaded hole 120 of a plate 100 at an angle of 10°. No modifications are required to the threaded hole 120 of the plate 100 in order to insert the variable-angle locking screw 200 at an angle to the threaded hole 120.

The system 001 described in this application achieves variable-angle insertion of locking screws by modifying the threads on the head of the locking screw and providing a plate 100 having standard threaded holes 120 with substantially continuous threads 121. Alternatively, variable-angle insertion of locking screws may be achieved by modifying the threaded holes 120 on the plate 100 and providing a locking screw having substantially continuous threads on the head. However, modifying the threaded holes on the plate to achieve variable-angle insertion of locking screws may be disadvantageous because such modifications typically require the removal of material from the plate, which may weaken the plate.

A system which is able to achieve variable-angle insertion of locking screws using a plate 100 having standard threaded holes 120 with substantially continuous threads 121 may provide increased flexibility compared to systems which achieve variable-angle insertion of locking screws by modifying the threaded holes on the plate. In a system which uses a plate 100 having standard threaded holes 120, the plate 100 is more versatile because any threaded hole 120 on the plate 100 is capable of receiving a standard locking screw 400 or a variable-angle locking screw 200. If a variable-angle locking screw 200 is selected, it may be inserted into any threaded hole 120 either coaxially or at an angle. Furthermore, if coaxial insertion is desired, a standard locking screw 400 may be selected to increase engagement between the locking screw and the plate 100, which may increase engagement between the threaded hole and the head of the locking screw. In contrast, if the threaded holes on the plates had discontinuous threads, increasing engagement between the plate and the locking screw during coaxial insertion would be more difficult because it would involve modifying an existing plate or custom-designing a plate with some threaded holes specifically designed for coaxial insertion.

The foregoing description is provided to enable any person skilled in the art to practice the various example implementations described herein. Various modifications to these variations will be readily apparent to those skilled in the art, and the generic principles defined herein may be applied to other implementations. All structural and functional equivalents to the elements of the various illustrious examples described throughout this disclosure that are known or later come to be known to those of ordinary skill in the art are expressly incorporated herein by reference.

What is claimed is:

1. A variable-angle locking (VAL) screw insertable into a threaded hole, the VAL screw comprising:
a head having a first end, a second end, and an outer surface extending between the first end and the second end; and
a shank extending from the first end of the head along a longitudinal axis of the VAL screw,
wherein the outer surface of the head includes a plurality of thread segments,
wherein the outer surface of the head includes engaging portions that engage the threaded hole and non-engaging portions that do not engage the threaded hole, and
wherein the plurality of thread segments extend across the engaging portions and do not extend across the non-engaging portions.

2. The VAL screw of claim 1, wherein the engaging portions and the non-engaging portions alternate around the outer surface of the head.

3. The VAL screw of claim 1, wherein the head comprises at least three engaging portions and at least three non-engaging portions.

4. The VAL screw of claim 1, wherein a curve extends along a trajectory formed by a crest of a first thread segment on a first engaging portion, and
wherein the curve follows the trajectory across a non-engaging portion and also extends along a trajectory formed by a crest of a second thread segment on an adjacent engaging portion.

5. The VAL screw of claim 1, wherein each non-engaging portion extends from the first end of the head to the second end of the head.

6. The VAL screw of claim 1, wherein each engaging portion comprises at least one thread segment.

7. The VAL screw of claim 1, wherein each engaging portion comprises a column formed by at least two of the plurality of thread segments.

8. The VAL screw of claim 1, wherein the engaging portions, when added together, cover at least about 25% of the outer surface of the head.

9. The VAL screw of claim 1, wherein the engaging portions, when added together, cover at least about half of the outer surface of the head and less than the entire outer surface of the head.

10. The VAL screw of claim 1, wherein a border between an engaging portion and an adjacent non-engaging portion is substantially coplanar with the longitudinal axis of the VAL screw.

11. A bone plate system comprising:
a plate having a threaded hole, wherein the threaded hole comprises a substantially continuous thread extending about an axis of the threaded hole; and
the VAL screw of claim 1,
wherein the VAL screw is insertable into the threaded hole of the plate.

12. The system of claim 11, wherein the VAL screw is insertable into the threaded hole of the plate at an angle ranging from about 0° to about 20°, wherein the angle is measured between the longitudinal axis of the VAL screw and the axis of the threaded hole.

13. The system of claim 11, further comprising a standard locking screw including:
a head having a first end, a second end, and an outer surface extending between the first end and the second end; and
a shank extending from the first end of the head along a longitudinal axis of the VAL screw,
wherein the outer surface of the head of the standard locking screw comprises one or more substantially continuous threads.

14. The system of claim 13, wherein the VAL screw and the standard locking screw are interchangeably insertable into the threaded hole of the plate.

15. The system of claim 13, wherein the plate comprises a plurality of threaded holes, and wherein each of the threaded holes interchangeably receives the VAL screw and the standard locking screw.

16. A variable-angle locking (VAL) screw insertable into a threaded hole, the VAL screw comprising:
a head having a first end, a second end, and an outer surface extending between the first end and the second end; and
a shank extending from the first end of the head along a longitudinal axis of the VAL screw,
wherein the outer surface of the head comprises one or more discontinuous threads.

17. The VAL screw of claim 16, wherein the outer surface of the head includes engaging portions that engage the threaded hole and non-engaging portions that do not engage the threaded hole.

18. The VAL screw of claim 17, wherein the engaging portions and the non-engaging portions alternate around the outer surface of the head.

19. The VAL screw of claim 17, wherein each non-engaging portion extends from the first end of the head to the second end of the head.

20. The VAL screw of claim 17, wherein the head comprises at least three engaging portions and at least three non-engaging portions

21. The VAL screw of claim 17, wherein each of the one or more discontinuous threads forms a plurality of thread segments, and
wherein the plurality of thread segments extend across the engaging portions and do not extend across the non-engaging portions.

22. The VAL screw of claim 17, wherein each engaging portion comprises at least one thread segment.

23. The VAL screw of claim 17, wherein each engaging portion comprises a column formed by at least two of the plurality of thread segments.

24. The VAL screw of claim 17, wherein the engaging portions, when added together, cover at least about 25% of the outer surface of the head.

25. The VAL screw of claim 17, wherein the engaging portions, when added together, cover at least about half of the outer surface of the head and less than the entire outer surface of the head.

26. The VAL screw of claim 17, wherein a border between an engaging portion and an adjacent non-engaging portion is substantially coplanar with the longitudinal axis of the VAL screw.

27. The VAL screw of claim 17, wherein a curve extends along a trajectory formed by a crest of a first thread segment on a first engaging portion, and
wherein the curve follows the trajectory across a non-engaging portion and also extends along a trajectory formed by a crest of a second thread segment on an adjacent engaging portion.

28. A bone plate system comprising:
a plate having a threaded hole, wherein the threaded hole comprises a substantially continuous thread extending about an axis of the threaded hole; and
the VAL screw of claim 16,
wherein the VAL screw is insertable into the threaded hole of the plate.

29. The system of claim 28, wherein the VAL screw is insertable into the threaded hole of the plate at an angle ranging from about 0° to about 20°, and wherein the angle is measured between the longitudinal axis of the VAL screw and the axis of the threaded hole.

30. The system of claim 28, further comprising a standard locking screw including:
a head having a first end, a second end, and an outer surface extending between the first end and the second end; and
a shank extending from the first end of the head along a longitudinal axis of the VAL screw,
wherein the outer surface of the head of the standard locking screw comprises one or more substantially continuous threads.

31. The system of claim 30, wherein the VAL screw and the standard locking screw are interchangeably insertable into the threaded hole of the plate.

32. The system of claim 30, wherein the plate comprises a plurality of threaded holes, and wherein each of the threaded holes interchangeably receives the VAL screw and the standard locking screw.
